(19) United States
(12) Patent Application Publication
(10) Pub. No.: US 2006/0058680 A1
(43) Pub. Date: Mar. 16, 2006

(54) Needle guide for laparoscopic ultrasonography

(76) Inventor: Stephen Solomon, New York, NY (US)

Correspondence Address: FRISHAUF, HOLTZ, GOODMAN & CHICK, PC, 220 Fifth Avenue, 16TH Floor, NEW YORK, NY, 10001-7708, US

(21) Appl. No.: 11/213,033

(22) Filed: Aug. 25, 2005

Related U.S. Application Data
(60) Provisional application No. 60/604,286, filed on Aug. 25, 2004.

(51) Int. Cl. A61B 8/14

(52) U.S. Cl. 600/466

(57) ABSTRACT

A guide device is provided for a laparoscopic ultrasound probe. The guide device includes a sheath that is adapted to fit over at least a portion of the ultrasound probe, and a guide coupled to the sheath for guiding a medical tool inserted therethrough to position the medical tool in a plane of an ultrasound image obtained via the ultrasound probe.

### CROSS-REFERENCE TO RELATED APPLICATION

This application claims the benefit of priority of provisional application Ser. No. 60/604,286, filed Aug. 25, 2004, the entire contents of which are incorporated herein by reference.

### FIELD OF THE INVENTION

The present invention relates to a medical tool guide for use with a laparoscopic ultrasound probe.

### BACKGROUND OF THE INVENTION

Laparoscopic ultrasonography is a known technique for visualizing regions inside of organs. The image obtained through laparoscopic ultrasonography is essentially a two-dimensional image extending from a transducer at the distal end of a laparoscopic ultrasound probe. Frequently, laparoscopic ultrasonography is used to visualize masses for needle biopsy or tumor ablation. To perform the biopsy or ablation, a needle or probe (or other instrument, referred to in this section as a “needle” for convenience) must be inserted into the mass while the mass is visualized in the ultrasound image. To accomplish this, the needle is kept in the plane of the ultrasound image while guiding the needle to the mass. The needle, however, is operated independently of the laparoscopic probe and is inserted separately from the laparoscopic probe. It is therefore challenging to insert the needle in the plane of the ultrasound image. See Berber et al, “Laparoscopic ultrasonography and biopsy of hepatic tumors in 310 patients” The American Journal of Surgery 187 (2004) 213-218, for a description of laparoscopic ultrasonography.

### OBJECT OF THE PRESNET INVENTION

It is an object of the present invention to provide a medical tool guide device for a laparoscopic ultrasound probe that enables adjustable positioning of the medical tool in the imaging plane.

### SUMMARY OF THE INVENTION

According to one aspect of the present invention a guide device is provided for a laparoscopic ultrasound probe. The guide device includes a sheath that is adapted to fit over at least a portion of the ultrasound probe, and a guide coupled to the sheath for guiding a medical tool inserted therethrough to position the medical tool in a plane of an ultrasound image obtained via the ultrasound probe.

### BRIEF DESCRIPTION OF THE DRAWINGS

FIG. 1 shows a guide device according to the present invention;

FIG. 2 shows a laparoscopic ultrasound probe inserted in the guide device of FIG. 1;

FIG. 3 shows the laparoscopic ultrasound probe inserted in the guide device in use to visualize a mass;

FIG. 4 shows a needle being introduced through the guide device of the present invention;

FIGS. 5A through 5D show the adjustment of the depth of the needle with respect to the ultrasound transducer using the guide device according to the present invention; and

FIG. 6 shows a modification of the guide device of the present invention.

### DETAILED DESCRIPTION

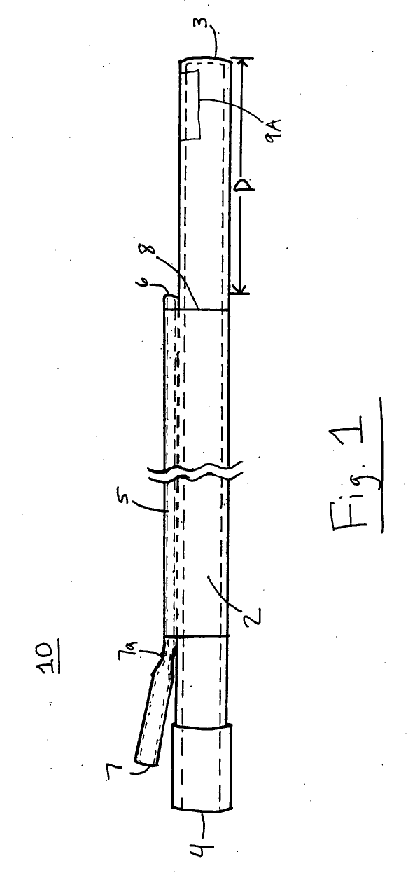

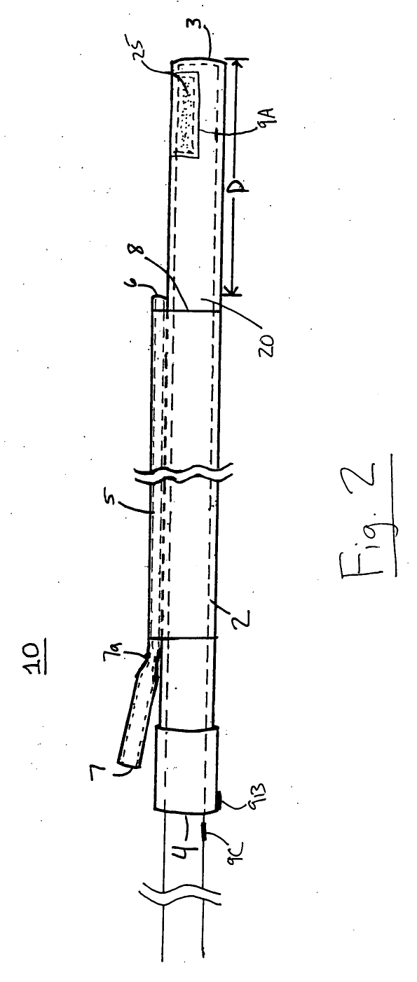

FIG. 1 shows a guide device 10 of the present invention, which includes a sheath 2 and a guide 5 fixed to the sheath 2. As shown in FIG. 2, the sheath 2 slides over the distal end of a laparoscopic ultrasound probe 20, such that the distal end of the probe 20 contacts the closed distal end 3 of the sheath 2 and the proximal portion of the probe 20 extends out through the open proximal end 4 of the sheath 2. The sheath 2 should fit snugly enough on the probe 20 that air is prevented from coming between the probe 20 and the sheath 2. When the probe 20 is laparoscopically inserted in the body of the patient, the proximal end 4 of the sheath 2 preferably remains outside the patient. According to the present invention, the guide is made from, for example, PVC or polyurethane or another plastic.

The guide 5 is attached to the sheath 2 to guide a medical tool (instrument), such as a biopsy needle or ablation probe inserted through the introduction port 7 and out through the distal end 6. As shown in FIG. 1, the introduction port 7 may be angled slightly with respect to the sheath 2 (and the probe 20 therein) to facilitate entry of the needle into the guide 5. See bent portion 7a in FIGS. 1 and 2. The angle of the introduction port 7 with respect to the sheath 2 should be small enough to allow a rigid or substantially rigid medical tool to pass therethrough. If the introduction port 7 is angled too much, a rigid or substantially rigid medical tool will not be able to pass around the bent portion 7a between the port 7 and the rest of the guide 5.

The guide 5 has a wide enough passageway (channel or lumen) to accommodate either a needle or an ablation probe, or another desired instrument. Since a radiofrequency ablation probe may be 15 gauge, the lumen of the guide 5 should preferably be larger than 15 gauge.

As shown in FIG. 1, the guide 5 is in the form of a tube and may be attached to the sheath 2 by a belt 8 that wraps tightly around both the sheath 2 and the guide 5 and that is attached to the sheath 2 with an adhesive. The belt 8 could also be adhered to both the sheath 2 and the guide 5. In addition, multiple smaller belts could be used instead of the large belt 8 shown in FIG. 1. Alternatively, the guide 5 could simply be fixed to the sheath 2 by adhesive, or the guide 5 and sheath 2 could be formed integrally.

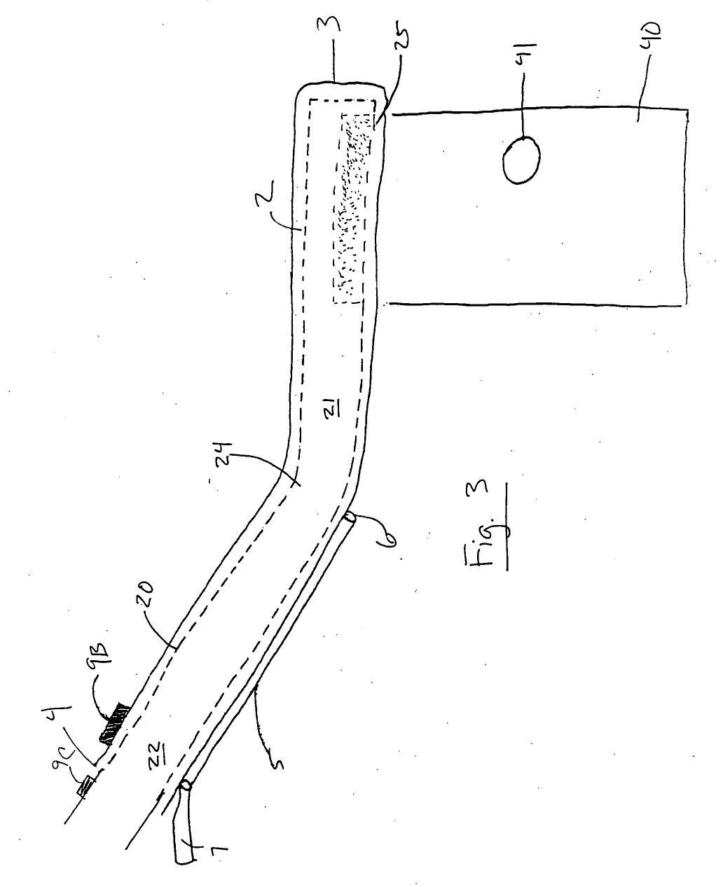

The laparoscopic ultrasound probe 20 is flexible so that the distal end thereof can be controlled to bend. See, for example, FIG. 3, in which the distal end portion 21 is bent with respect to the proximal end portion 22 of the probe 20 at a bending section 24. Such laparoscopic ultrasound probes are known in the art and, as described in Berber et al (supra), may be controlled by knobs in a similar manner as a flexible endoscope to allow about the distal 5 cm thereof, for example, to be bent with respect to the rest of the probe.

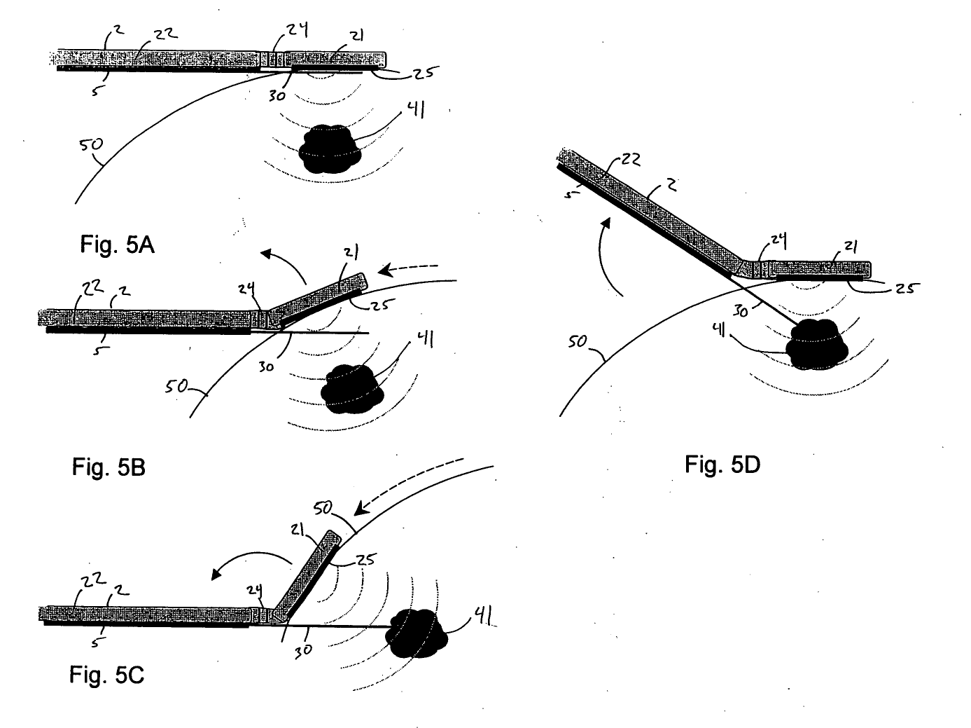

To allow substantially rigid or rigid medical tools to be inserted through the guide 5 while the probe 20 is bent, the guide 5 is set back from the distal end 3 of the sheath 2 by a distance D (see FIG. 1) that is at least as long as the distance from the distal end 3 of the probe 20 to the point at which the probe 20 bends in the bending section 24. For example, the distal end 6 of the guide 5 is provided at the distal end of the proximal end portion 22 of the probe 20 (see FIGS. 5A-5D, for example).

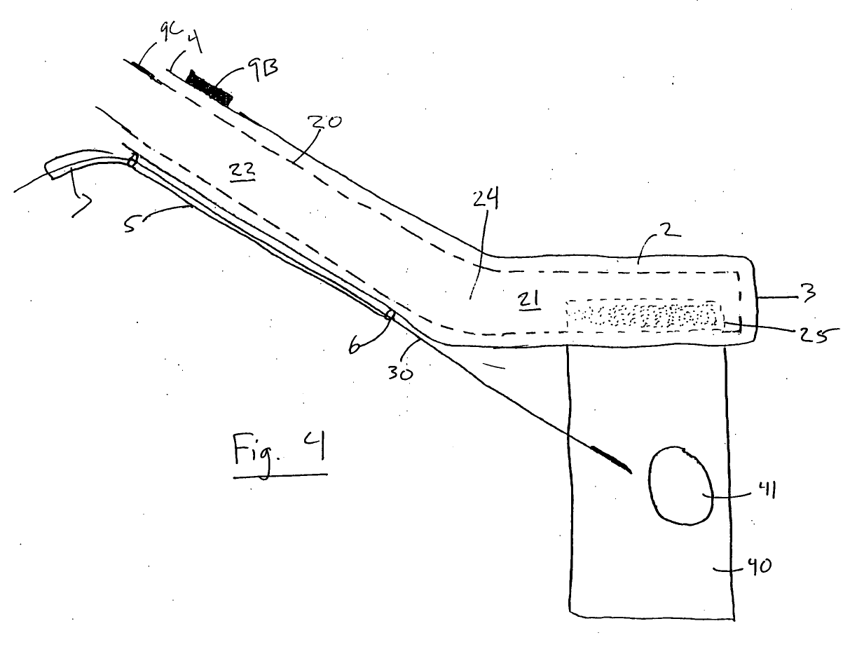

As shown in FIG. 4, a medical tool 30 is inserted through the guide 5, which is positioned so that the medical tool 30 introduced therethrough is visible in the imaging plane 40 of the ultrasound image obtained via the ultrasound transducer 25 located at the distal end portion 21 of the probe 20. The probe 20 is bent by a desired amount to change the exiting angle of the medical tool 30 from the guide 5 with respect to the distal end portion 21 of the probe 20. The depth of the medical tool 30 in the imaging plane 40 may thereby be adjusted to target a mass 41 for biopsy or ablation with the medical tool 30.

FIGS. 5A-5D show the bending of the probe 20 to target the mass 41 with the medical tool 30. FIG. 5A shows the medical tool 30 exiting the guide 5 while the probe 20 is not bent. With this orientation of the probe 20, the medical tool 30 is immediately adjacent to the transducer 25. As shown in FIG. 5B, if the transducer 25 is moved along the organ 50 and the distal end portion 21 of the probe 20 is bent about the bending section 24, it is possible to reach a deeper location in the organ 50. FIG. 5C shows the transducer 25 moved further along the organ 50 with respect to FIG. 5B and the distal end portion 21 bent sufficiently to allow the needle to reach the mass 41 while both the medical tool 30 and the mass 41 are visualized via the ultrasound transducer 25. FIG. 5D shows the transducer 25 positioned at the same location on the organ 50 as in FIG. 5A. In FIG. 5D, the proximal end portion 22 of the probe 20 has been bent while the distal end portion 21 remains in place to allow the medical tool 30 to reach the mass 41 while visualizing both the medical tool 30 and the mass 41 via the ultrasound transducer 25.

As shown in FIG. 2 one or more markers may be provided on the guide device 10 to align the guide device 10 on the probe 20 to ensure that the imaging plane of the image obtained with the transducer 25 and the medical tool 30 introduced through the guide are co-located. For example, a marker 9A may be provided at the distal end of the sheath 2 as a box or other marker to be aligned with the ultrasound transducer 25. In addition, a marker 9B may be provided at the proximal end of the sheath 2 to be visible to an operator of the probe 20 while the probe 20 and guide device 10 are inserted in a patient. In this case, the marker 9B may be aligned with a marker 9C provided on the probe 20. The marker 9C may be a sticker that is removably attached to the probe 20 when the guide device 10 is fitted on the probe 20. For example, when the marker 9A is used to align the guide device 10 on the probe 20, the marker 9C may be fixed to the probe 20 in a position that is aligned with the marker 9B at the proximal end of the sheath 2. Thus, when the probe 20 and guide device 10 are inserted in the patient, it should be possible for the operator of the probe 20 to ensure that the guide device 10 is properly aligned even when the marker 9A is no longer visible.

According to the present invention, the laparoscopic ultrasound probe 20 may slide into the sheath 2, and the sheath is sufficiently tight to be secure on the probe 20. However, the guide device 10 according to the present invention may also be fixed to the sheath using a vacuum chamber to expand the sheath so that the probe 20 can be inserted in the sheath.

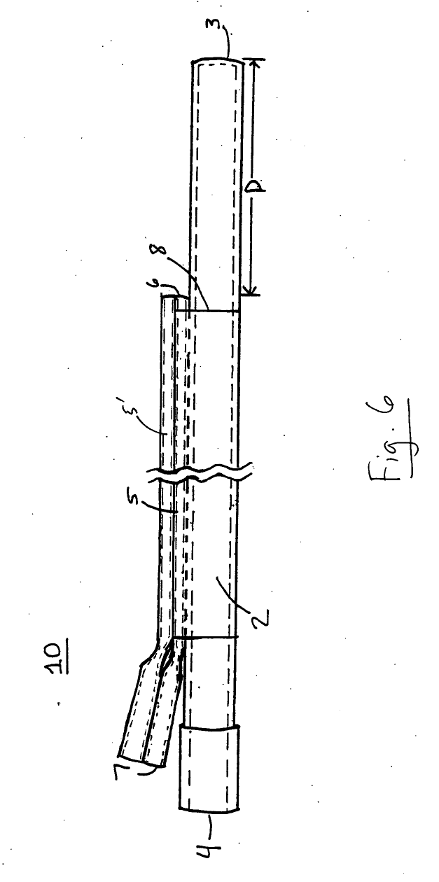

In addition, according to the present invention, multiple guide tubes could be attached to the sheath 2 to allow multiple medical tools to be placed simultaneously in the imaging plane 40 of the ultrasound transducer 25. For example, guides 5 and 5′ shown in FIG. 6 could be used to guide both a biopsy needle and a grasper to the imaging plane. The guides 5 and 5′ may be different sizes, for example to accommodate different types of medical tools.

As described hereinabove, the guide device 10 according to the present invention may be slidably fitted onto a laparoscopic ultrasound probe 20 to allow the medical tool 30 to be reliably introduced into the imaging plane 40 visualized via the ultrasound transducer 25. Since the probe 20 is flexible, and the guide 5 is set back from the distal end of the probe 20 so as not to interfere with the flexibility thereof, the exiting angle of the medical tool 30 from the guide 5 may be adjusted by bending the probe. The depth of penetration of the medical tool 30 in the imaging plane 40 in the organ 50 may thereby be easily adjusted. In addition, since the guide device 10 according to the present invention covers the portion of the laparoscopic ultrasound probe 20 inserted in the body of the patient, the guide device 10 may provide advantages in the sterility of laparoscopic ultrasonography and may facilitate clean-up after laparoscopic ultrasonography procedures.

The foregoing provides a detailed description of presently preferred embodiments. Various modifications and additions can be made without departing from the spirit and scope of the invention. In particular, the terms “needle” and “medical tool” as used herein should be understood to include any medical device that could be inserted through the guide 5 of the present invention, such as a biopsy needle, ablation probe, grasper or other instrument. In addition, while the guide 5 and lumen therethrough are shown in the drawings as being round, other shapes of guides and lumens may, of course, be used. Accordingly, the foregoing description is meant to be taken only by way of example and not to otherwise limit the scope of the present invention as defined in the appended claims.

What is claimed is:

1. A guide device for a laparoscopic ultrasound probe, said guide device comprising:
a sheath that is adapted to fit over at least a portion of the ultrasound probe; and
a guide coupled to the sheath for guiding a medical tool inserted therethrough to position the medical tool in a plane of an ultrasound image obtained via the ultrasound probe.

2. The guide device according to claim 1, wherein the guide comprises a tube having a lumen therethrough.

3. The guide device according to claim 2, wherein the lumen is at least 15 gauge.

4. The guide device according to claim 1, wherein the ultrasound probe is bendable, and an exiting angle of the medical tool from the guide with respect to a distal end portion of the ultrasound probe is adjustable by bending the ultrasound probe.

5. The guide device according to claim 4, wherein a distance from a distal end of the sheath to a distal end of the guide is not less than a distance from a distal end of the ultrasound probe to a bending portion about which the ultrasound probe is bendable.

6. The guide device according to claim 1, further comprising at least one marker for aligning the guide with the plane of the ultrasound image.

7. The guide device according to claim 6, wherein the marker is positioned at a distal end of the sheath to be aligned with an ultrasound transducer of the ultrasound probe.

8. The guide device according to claim 6, wherein the at least one marker comprises:
a first marker provided at a proximal end of the sheath; and
a second marker provided on the ultrasound probe to be aligned with the marker at the proximal end of the sheath.

9. The guide device according to claim 1, further comprising an additional guide for guiding an additional medical tool inserted therethrough to position the additional medical tool in the plane of the ultrasound image.

10. The guide device according to claim 9, wherein the additional guide is coupled to the guide that is fixed to the sheath.

11. The guide device according to claim 2, wherein the guide tube comprises an introduction port at a proximal end thereof, and the introduction port is angled with respect to the sheath.

12. The guide device according to claim 11, wherein an interior of the introduction port is shaped as a funnel

13. A guide device for an ultrasound probe, said device comprising:
a sheath that is adapted to fit over at least a portion of the ultrasound probe;
a guide coupled to the sheath for guiding a medical tool inserted therethrough to position the medical tool in a plane of an ultrasound image obtained via the ultrasound probe; and
at least one marker for aligning the guide with the plane of the ultrasound image;
wherein the ultrasound probe is bendable, and an exiting angle of the medical tool from the guide tube with respect to a distal end portion of the ultrasound probe is adjustable by bending the ultrasound probe.
